(19) United States
(12) Patent Application Publication
(10) Pub. No.: US 2016/0331956 A1
(43) Pub. Date: Nov. 17, 2016

(54) METHOD AND APPARATUS FOR MINIMALLY INVASIVE IMPLANTABLE MODULATORS

(71) Applicant: Nalu Medical, Inc., Carlsbad, CA (US)

(72) Inventors: Anatoly Yakovlev, Santa Clara, CA (US); Daniel Pivonka, Palo Alto, CA (US)

(21) Appl. No.: 14/975,358

(22) Filed: Dec. 18, 2015

Related U.S. Application Data
(63) Continuation of application No. PCT/US2014/043023, filed on Jun. 18, 2014.
(60) Provisional application No. 61/836,536, filed on Jun. 18, 2013.
(60) Provisional application No. 61/836,544, filed on Jun. 18, 2013.

(51) Int. Cl. A61N 1/05; A61B 5/04; A61N 1/378

(52) U.S. Cl. CPC A61N 1/0556; A61N 1/3787; A61B 5/04001

(57) ABSTRACT

Some embodiments of the present invention provide an apparatus for providing therapies and/or diagnostics with an implanted system. Some embodiments of the present invention include methods and apparatus for modulating tissues with conventional methods and/or new methods using mechanical forces. Some embodiments of the present invention include methods and apparatus for minimally invasive delivery of implanted systems. Some embodiments of the present invention include methods and apparatus for extensions of the implanted system that can expand, unroll, unfold, and/or unfurl.

### CROSS-REFERENCE TO RELATED APPLICATIONS

This application is a continuation of PCT Application No. PCT/US2014/043023 (Attorney Docket No. 47476-703.601), filed Jun. 18, 2014, which claims priority to U.S. Provisional Application Ser. No. 61/836,536, entitled “Method and Apparatus for Implantable Mechanical Neuromodulation Devices”, filed Jun. 18, 2013, and U.S. Provisional Application Ser. No. 61/836,544, entitled “Method and Apparatus for Needle Injectable Implantable Devices”, filed Jun., 18, 2013, the content of each of which is incorporated herein by reference in their entirety.

### FIELD OF THE INVENTION

The present invention relates to methods of making and using and apparatus for mechanically modulating nerves inside the body, particularly for long-term use in low power miniaturized implantable devices. The present invention also relates to methods of making and using an apparatus for minimally invasive implantable devices for therapeutic and/or diagnostic purposes.

### BACKGROUND OF THE INVENTION

Implantable medical devices are used for a number of different conditions and therapies. Many implantable medical devices are large in size, because they rely on batteries as an energy source. Neuromodulation devices, such as pacemakers, also have long leads. Leads have electrodes at one end, which are placed next to the stimulation site and are connected to the pacemaker, which is implanted separately under the skin. The batteries have to be replaced every several years. These devices require invasive implantation and suffer from poor power efficiency, due to the nature of the electrical stimulation methods. Additionally, these methods gradually become less effective, because the electrical contact with the tissue degrades over time.

Also, these methods can potentially cause nerve damage, because of the high voltages and currents that are applied.

There are also known methods for wirelessly communicating data and providing power, particularly from a location exterior to a body and to an implantable device disposed within a body of tissue. One such discussion is provided in the patent application entitled “Method and Apparatus for Efficient Communication with Implantable Devices” filed as U.S. patent application Ser. No. 13/734,772, on Jan. 4, 2012, the full disclosure of which is hereby incorporated by reference.

Nerve stimulation treatments have shown increasing promise, showing potential in the treatment of many chronic diseases, including drug-resistant hypertension, motility disorders in the intestinal system, metabolic disorders arising from diabetes and obesity, and chronic pain conditions among others. Many of these treatments have not been developed effectively, because of the lack of miniaturization and power efficiency, in addition to other factors.

Wirelessly powered systems with communication are desirable, because they can be miniaturized and have no need for battery replacements. However, wireless systems have an even more restrictive power budget.

### BRIEF SUMMARY

According to one aspect of the present inventive concepts, an apparatus for modulating tissue comprises an implanted system comprising a tissue modulator. The tissue modulator can be constructed and arranged to induce a physiological response from tissue with mechanical forces.

In some embodiments the tissue comprises nerve tissue.

In some embodiments the tissue comprises muscle tissue.

In some embodiments the implanted system comprises at least one active component.

In some embodiments the implanted system comprises at least one passive component.

In some embodiments the implanted system is constructed and arranged to harvest energy from the environment surrounding the implanted system. The implanted system can be constructed and arranged to harvest energy selected from the group consisting of: heat energy; motion energy; and combinations thereof.

In some embodiments the implanted system further comprises a power receiver constructed and arranged to receive power transmitted transcutaneously to the implanted system. The power receiver can be constructed and arranged to receive power selected from the group consisting of: radio frequency energy; ultrasound energy; and combinations thereof.

In some embodiments the implanted system further comprises a power supply. The power supply can comprise a component selected from the group consisting of: a battery; a capacitor; and combinations thereof.

In some embodiments the apparatus further comprises a housing surrounding at least one portion or the entirety of the tissue modulator.

In some embodiments the tissue modulator comprises a mechanical effector constructed and arranged to physically engage a nerve and an electromagnetic actuator coupled to the mechanical effector, and the electromagnetic actuator is constructed and arranged to induce motion, induce rotation, change a dimension, and/or change a shape of the mechanical effector in a way that would modulate the nerve.

In some embodiments the tissue modulator comprises a mechanical effector constructed and arranged to directly and/or indirectly engage a nerve and a coil constructed and arranged to cause movement of the mechanical effector. The apparatus can further comprise an external magnetic source constructed and arranged to magnetically couple to at least an implanted portion of the tissue modulator.

In some embodiments the tissue modulator comprises an electromagnetic transducer. The electromagnetic transducer can be configured to generate a constant magnetic field that generates the mechanical forces. The electromagnetic transducer can be configured to generate a varying magnetic field that generates the mechanical forces.

In some embodiments the tissue modulator comprises a magnetic transducer. The magnetic transducer can comprise a passive magnet. The magnetic transducer can comprise an electromagnet.

In some embodiments the tissue modulator comprises at least one actuator constructed and arranged to deliver the mechanical forces to tissue and/or modulate a nerve. The actuator can comprise a piezoelectric based actuator. The actuator can comprise a thermal actuator. The actuator can comprise a motor. The actuator can comprise a linear actuator. The actuator can comprise a vibrating beam. The actuator can be controlled by varying a parameter of the applied force selected from the group consisting of: amplitude; frequency; duty cycle; duration; and combinations thereof. The actuator can be controlled by varying a magnetic field. The actuator can be controlled by varying current.

In some embodiments the apparatus further comprises an external system. The external system can be configured to control the implanted system and/or the tissue modulator. The external system can be configured to transmit data to the implanted system and/or the tissue modulator. The external system is configured to transmit power transcutaneously to the implanted system and/or the tissue modulator. The power transmitter is constructed and arranged to transmit power selected from the group consisting of: radiofrequency energy; ultrasound energy; and combination thereof. The external system can be configured to magnetically couple to the implanted system and/or the tissue modulator. The magnetic coupling can be configured to cause movement of the tissue modulator to induce the physiological response from tissue.

According to another aspect of the present inventive concepts an therapeutic and/or diagnostic implant for inducing a physiologic response in tissue and/or monitoring tissue activity comprises a body portion for implanting with a patient and at least one extension attached to the body portion. The extension is constructed and arranged to transition from a compacted state to an expanded state after the body portion is implanted in the patient.

In some embodiments the therapeutic and/or diagnostic implant is constructed and arranged to perform a function selected from the group consisting of: delivering energy to tissue; delivering therapy to tissue; delivering pharmaceutical agent to tissue; modulating tissue; sensing physiological and/or neural activity; sensing environmental conditions; sensing therapeutic outcomes and/or delivered therapy parameters; and combinations thereof.

In some embodiments the implant is constructed and arranged to be delivered through an introducer. The introducer can comprise a needle. The introducer can be less than 5 mm, 4 mm, 3 mm, 2 mm, or 1 mm in diameter. The introducer can comprise a flexible cannula and a removable rigid cannula slidingly received by the flexible cannula.

In some embodiments transitioning from the compacted state to the expanded state comprises a physical change selected from the group consisting of: unfolding; unrolling; unfurling; and combinations thereof.

In some embodiments the at least one extension comprises multiple extensions constructed and arranged to unfold, unroll and/or unfurl.

In some embodiments the extension comprises an energy harvesting element.

In some embodiments the extension comprises an antenna. The antenna can comprise an RCS that increases as the extension transitions from the compacted state to the expanded state.

In some embodiments the extension comprises an electrode.

In some embodiments the extension comprises an energy storage element.

In some embodiments the extension comprises at least a flexible portion.

In some embodiments the extension comprises at least a conformable portion. The extension can comprise a conformable electrode. The conformable electrode can be constructed and arranged to at least partially surround a curved segment of tissue to be modulated and/or sensed.

In some embodiments the extension comprises two sliding beams. The extension can comprise a cuff electrode, and the compacted state comprises a relatively straight configuration and the expanded state comprises a ring configuration.

In some embodiments the extension is constructed and arranged to be removed from the body portion.

In some embodiments the extension is constructed and arranged to be detachable from the body portion.

In some embodiments the extension comprises a spring portion resiliently biased to expand as the extension transitions from the compacted state to the expanded state. The extension can comprise a tweezer-like construction.

In some embodiments the extension comprises a flexible substrate constructed and arranged to unroll as the extension transitions from the compacted state to the expanded state.

In some embodiments the extension comprises an inflatable portion constructed and arranged to expand when filled with fluid. The fluid can comprise a material selected from the group consisting of: a gas; a liquid; a gel; and combinations thereof. In some embodiments the fluid may be liquid, such as saline.

In some embodiments the extension comprises an anchor element. The anchor element can comprise an element selected from the group consisting of: threads; arrow-shaped element; harpoon-shaped element; and combinations thereof. The anchor element can comprise threads. The therapeutic and/or diagnostic implant can further comprise an introducer for delivering the body portion within the patient.

In some embodiments the extension is constructed and arranged to be replaced after implantation in the patient.

In some embodiments the therapeutic and/or diagnostic implant further comprises a coating. The coating can be positioned on a location selected from the group consisting of: body portion; extension; and combinations thereof. The coating can comprise at least one of a dissolvable coating or a biodegradable coating. The coating can comprise a drug-eluding agent.

In some embodiments the therapeutic and/or diagnostic implant further comprises an introducer constructed and arranged to slidingly receive the body portion and the extension and to deliver the body portion and the extension within the patient. The therapeutic and/or diagnostic implant can further comprise an imaging element. The imaging element is selected from the group consisting of: an optical fiber; a camera; and combinations thereof.

In some embodiments the therapeutic and/or diagnostic implant further comprises an extraction tool constructed and arranged to extract the therapeutic and/or diagnostic implant from the patient. The extraction tool further comprises an imaging element. The imaging element is selected from the group consisting of: an optical fiber; a camera; and combinations thereof.

According to another aspect of the present inventive concepts, a method of delivering an therapeutic and/or diagnostic implant comprises delivering an therapeutic and/or diagnostic implant as described herein to a location within the patient; expanding the at least one extension after the delivery of the therapeutic and/or diagnostic implant into the patient; and inducing a physiological response from tissue proximate the therapeutic and/or diagnostic implant.

In some embodiments the therapeutic and/or diagnostic implant is delivered through an introducer. The therapeutic and/or diagnostic implant can exit the introducer while the introducer is being removed from the body of the patient.

In some embodiments the method further comprises performing a patient imaging procedure during the delivery of the therapeutic and/or diagnostic implant into the patient.

In some embodiments the method further comprises removing the therapeutic and/or diagnostic implant from the patient.

In some embodiments the method further comprises replacing the at least one extension of an implanted energy delivery element.

According to another aspect of the present inventive concepts, an injectable device comprises a structure sized or configured to be sized within a lumen of an introducer, with at least one active and/or passive component c coupled to the structure and configured to deploy at a target site within a patient body after the structure had been advanced from the introducer at the target site.

In some embodiments the at least one active component comprises a component selected from the group consisting of: an electrode; an antenna; an integrated and/or discrete circuit (IC); an energy storage element; and combinations thereof. The at least one active and/or passive component can comprise a pair of electrodes. The pair of electrodes can be fixed to the structure. At least one electrode of the pair of electrodes actively can deploy from the structure after the structure has been advanced from the introducer.

In some embodiments the at least one component comprises an antenna constructed and arranged to expand after being advanced from the introducer.

In some embodiments the antenna is fixed as a coil on the structure.

These and other aspects and embodiments will be described in further detail below, in reference to the attached drawing figures.

### BRIEF DESCRIPTION OF DRAWINGS

FIG. 1 illustrates an apparatus comprising an implanted system wirelessly coupled with a system external to the body, consistent with the present inventive concepts;

FIG. 2 illustrates a diagram of an active implanted system, consistent with the present inventive concepts;

FIG. 3 illustrates a minimally invasive implantable system delivered through a needle consistent with the present inventive concepts;

FIG. 4 illustrates a fully enclosed implant encapsulated to be delivered with minimally invasive methods, consistent with the present inventive concepts;

FIG. 5 illustrates a minimally invasive implantable system with an unfolding extension after implantation, consistent with the present inventive concepts;

FIGS. 6a-6c illustrate several embodiments of a minimally invasive device with unfolding or expanding extensions, consistent with the present inventive concepts;

FIG. 7 illustrates a minimally invasive device with expandable electrodes, consistent with the present inventive concepts;

FIG. 8 illustrates a minimally invasive device with harpoon or barbed electrodes, consistent with the present inventive concepts;

FIG. 9 illustrates a minimally invasive device with flexible electrodes that have barbed or harpoon tips for attaching to tissue, consistent with the present inventive concepts;

FIG. 10 illustrates a minimally invasive device with a screw electrode for attaching to tissue, consistent with the present inventive concepts;

FIG. 11 illustrates a minimally invasive device with a conformable attachment to a target tissue site, and the mechanism for this attachment, consistent with the present inventive concepts;

FIG. 12 illustrates an implementation of a mechanical modulator with an external magnetic field source/generator and an implanted magnetic material or implanted current-carrying wires, consistent with the present inventive concepts;

FIG. 13 illustrates an implementation of a mechanical modulator with an external electromagnet creating fields that influence an implanted magnetic material or current-carrying wires, consistent with the present inventive concepts;

FIG. 14 illustrates a mechanical actuator using an electromagnet to create mechanical forces for modulating tissues, consistent with the present inventive concepts;

FIG. 15 illustrates wire and current flow characteristics that experience forces or torques in the presence of a magnetic field, consistent with the present inventive concepts;

FIG. 16 illustrates a piezoelectric actuator for mechanically modulating tissues, consistent with the present inventive concepts.

### DETAILED DESCRIPTION OF THE INVENTION

Various embodiments described herein include a stimulation apparatus that includes an implantable portion, such as an implantable portion configured to stimulate tissue via mechanical actuation. Some embodiments include needle-injectable implants, such as needle injectable implants that comprise expandable (e.g. unfolding and/or unrolling) extensions. This disclosure also describes methods of making and using apparatus for needle injectable implantable devices for therapeutic and/or diagnostic purposes, which can also be capable of conventional electrical modulation or new types of mechanical modulation of tissues.

Also described herein are methods of making and using an apparatus for minimally invasive implantable devices for therapeutic and/or diagnostic purposes. A small form-factor for implants would allow for implantation surgery to be minimally invasive that can potentially be performed with a doctor's office procedure as opposed to a surgery. The small form factor can be achieved by packaging these devices into a shape that can be injected directly into the desired location within a body of tissue through a needle using an injection device similar to a syringe.

The needle injectable implant can be packaged in a sterile needle and packaged together with a disposable injection device. The needle and the injection device can be similar to a conventional needle and syringe and can be customized for a particular therapy application.

The injectable device may include a main body, which is similar in shape to the injection needle. The device may also include one or more optional extensions. Optional extensions can be antenna, electrodes, leads, or other extensions that also fit in the injection device and needle. These extensions are implanted together with the main body or can be delivered separately and assembled inside the body. Furthermore, the extensions can be made flexible to conform to anatomy. The optional extension would allow for the stimulation device and electrodes to be at a different depth from the tissue surface than the antenna, for example. In that case, the antenna may be positioned close to the tissue surface. This would allow for less tissue absorption when delivering energy to, or communicating with, the implant and thus improve link gain between the external antenna and the implanted device antenna.

The main body and the extensions can also be compacted in the needle and unfold, unroll and/or unfurl (hereinafter “unfold”) or otherwise expand (i.e. transition from a compacted state to an expanded state) in order to obtain a different shape after the implant is ejected from the needle. One reason for this expansion can be to make the antenna radar cross section (RCS) larger in order to capture more energy. This can be beneficial for deeper implants by enabling the antenna to harvest more energy and have better link gain. Another reason for expandable extension can be to position stimulation or sensing electrodes further apart. The expandable extensions and/or device are advantageous because they allow for use of smaller diameter needles and thus less invasive implantation procedure while still allowing for optimization of the device design.

Various embodiments also provide apparatus and methods for mechanical devices that modulate nerves with minimal power requirements. These devices can be miniaturized to millimeter and sub-millimeter scales, depending on the needs of the application. Additionally, performance does not degrade in the same way as electrical stimulation, because electrical contacts with tissue are not required. This allows these devices to be used with more sensitive nerves and tissues without causing damage. Also, because of the much lower voltage and current requirements, these methods are well-suited for fully wireless implants.

Mechanical forces can be generated from a wide variety of sources, including magnetic forces, electromagnetic forces, piezoelectric forces, or thermal expansion forces among others. These forces have different scaling properties and are most effective at different sizes. For maximum miniaturization and power efficiency, electromagnetic forces have significant potential for nerve stimulation. There are several embodiments that implement these forces. One implementation uses electric currents to create a magnetic field, which then manipulates either a permanent magnet or magnetic material. This method is similar to a miniaturized solenoid and can generate fairly significant forces. A variation of this method is to generate the magnetic field externally with either a magnet or an electromagnet so that the implanted device is entirely passive. In another implementation, a magnetic field is established and the mechanical force is generated from forces on current-carrying wires, which are very simple to construct and can be very small. Again, the magnetic field can be generated either internally or externally with either a magnet or electromagnet. Both implementations have flexibility in their construction and operation, which can be adapted to specific therapies.

In some embodiments, magnetic fields can be generated externally, to allow either passive or active devices to create mechanical force. The actuators can be interfaced with known remotely powered devices, battery powered devices, or passive setups; alternatively, they can be incorporated in the design of new devices.

For certain therapies, other mechanical methods may have advantages over electromagnetic forces because they have no need for a magnetic field. Typically, these other methods will be larger, with more limitations under the operating conditions, though may offer advantages in the magnitude of the force or they may be better suited to the environment.

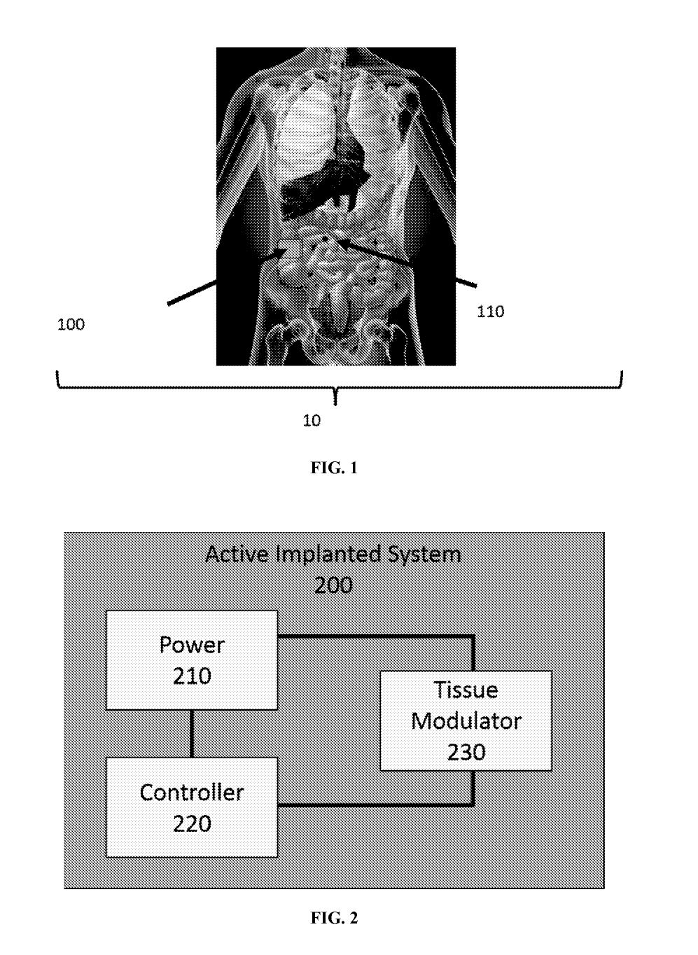

Piezoelectric methods can take the form of vibrating beams or motors, and can generate significant forces at specific frequencies. Thermoelectric actuators may also be useful, because they operate simply at small sizes. Implantable systems are limited in their design by the power budget, which restricts both miniaturization and functionality. Embodiments described herein include a minimally invasive implantable system with an external system FIG. 1 depicts apparatus 10 configured to stimulate tissue and comprising implantable system 110 and external system 100. Depending on the application, this implant can be placed in different locations inside the body and can include a variety of systems. This implant can be either active or passive (e.g. include one or more active or passive components, respectfully), and the external equipment can consist of power transmission, data transmission, and/or magnetic field generation. Also, depending on the application, there may be one or more implantable systems 110 coupled with a single or multiple external systems 100.

A diagram of another tissue stimulating active implant system 200, according to another embodiment, is shown in FIG. 2. Active implant 200 can comprise a power source 210, which can include a power receiver and/or a power supply. The power source 210 can be either locally powered such as with batteries, capacitors, etc, and/or by harvesting energy from the body (heat, motion and/or chemical energy, for example) and/or remotely powered, such as with RF energy (near-, far-, or mid-field powering) or ultrasound. The controller 220 can be designed to work with existing devices, and can allow for either analog or digital control of the modulator, power systems, or other components. Controller 220 can also manage incoming data and communications with other systems. Data for controlling the stimulation profile can be received from an external transmitter (e.g. external system 100 of FIG. 1). Implanted system 200 can comprise a tissue modulator 230, which can be surrounded by a housing (e.g. a sealing housing) surrounding at least a portion of tissue modulator 230. The modulator 230 can employ any of the active methods that are described herein, including conventional electrical methods and new methods for mechanical modulation as described herein. Modulator 230 may include an electromagnetic transducer such as a tranducer through which current flows, to generate a constant and/or varying magnetic field. Modulator 200 may include a magnetic transducer, such as a transducer comprising a passive magnet and/or an electromagnet. Modulator 230 may include an actuator that delivers mechanical force to tissue, such as an actuator selected from the group consisting of: a piezoelectric-based actuator; a thermal actuator; a motor; a linear actuator; a vibrating beam; and combinations of these. Modulator 230 may include an actuator that is controlled by varying one or more of: amplitude; frequency; duty cycle; and duration.

Modulator 230 can be controlled by varying a magnetic field and/or by varying current.

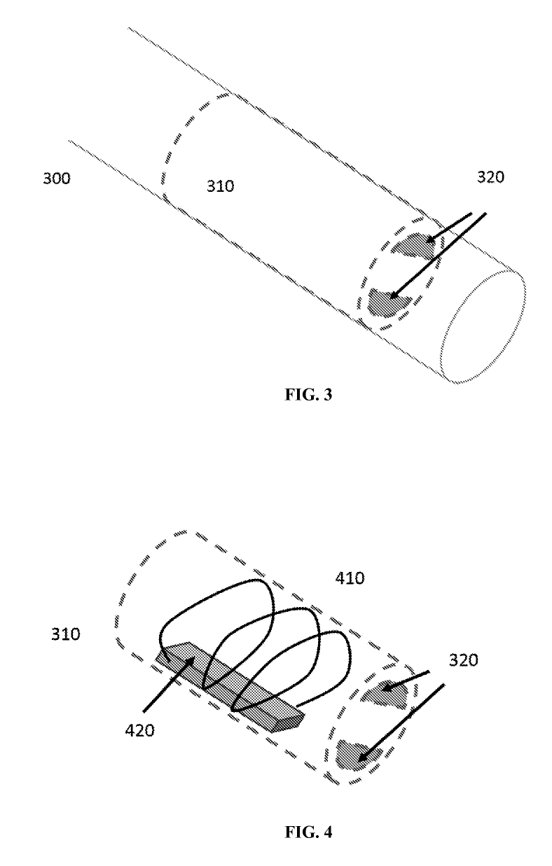

In various embodiments, the implant portion of the system may perform any of a number of desired goals of diagnosis, therapy, or both. A major advantage of the implants described herein is that they are miniaturized and packaged such that they can be easily implanted using minimally invasive methods, such as via injection through a needle, for example, without the need for surgery, as shown in FIGS. 3 and 4. The implant main body 310 can fit inside of a delivery needle or introducer 300. The implant main body 310 can also include one or more electrodes 320 that interface with the tissue or environment for modulation or sensing. The implant body 310 can also contain one or more fully enclosed antennas 410 and one or more integrated circuits 420 and other discrete passive or active components. The electrodes 320 can again interface with the tissue or environment.

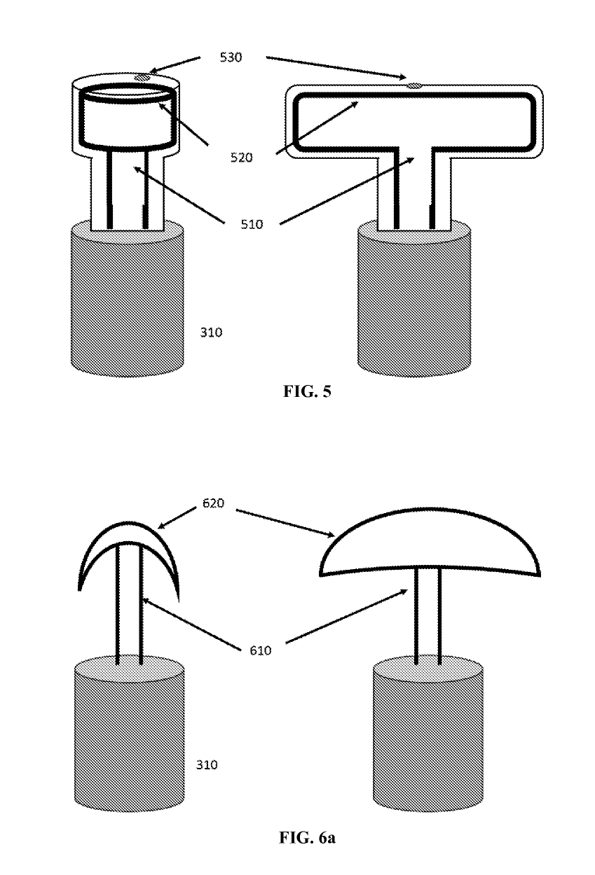

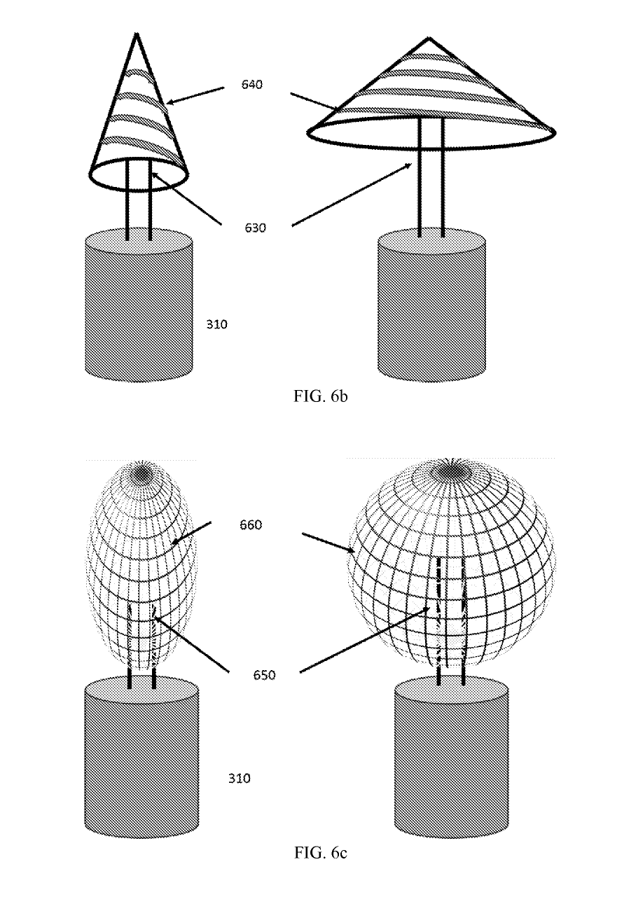

The device itself can be completely contained in a non-deformable package or contain a rigid body with one or more extensions similar to 510 that can be made unfoldable, expandable, or detachable for form-factor or shape adaptation during or after the implantation procedure as shown in FIG. 5 and FIGS. 6a-6c. Methods for delivering different types of unfolding or expanding extensions are illustrated in 510, 610, 630, and 650.

In these figures, the flexible extensions 510, 610, 630, and 650 are illustrated in compact form on the left side and in expanded form on the right side of the figures. Some embodiments may include a rolled up extension which forms a rectangular antenna substrate 510, a parachute-like structure 610, an umbrella-like structure 630, and/or a spherical antenna structure 650. These structures may include printed conductor traces 520, 620, 640, and 660, which may form one or more planar loop, spiral, or dipole antennas when unfolded or inflated after implantation. For very small device sizes, the delivered power to the device can be very limited, especially for deeply implanted devices. Therefore, it may be beneficial to implement the antenna on a flexible substrate that can be folded (or rolled) in a way to make the device injectable with a needle. Examples of unfolding or expanding antennas are shown in 520, 620, 640, and 660. After the injection, the antenna can be made to unfold (or unroll) such as to make its radar cross section (RCS) larger and thus increase the amount of power it can harvest.

Additionally, the antenna only or antenna together with the energy harvesting circuitry (such as matching network and rectifier) can be made separate from the rest of the implant in a form of unfolding extension. This would allow for the antenna to be closer to the tissue surface and therefore would reduce separation distance between the external transmitter and the implant antenna and would therefore potentially improve the link gain. Having rectification circuitry close to the antenna can be advantageous to maintain reasonably high quality factor that is not limited by the transmission line losses. Alternatively, the transmission line can serve as part of the matching network. Some sketches for the implantation of various needle injectable devices are shown in FIG. 5 and FIG. 6.

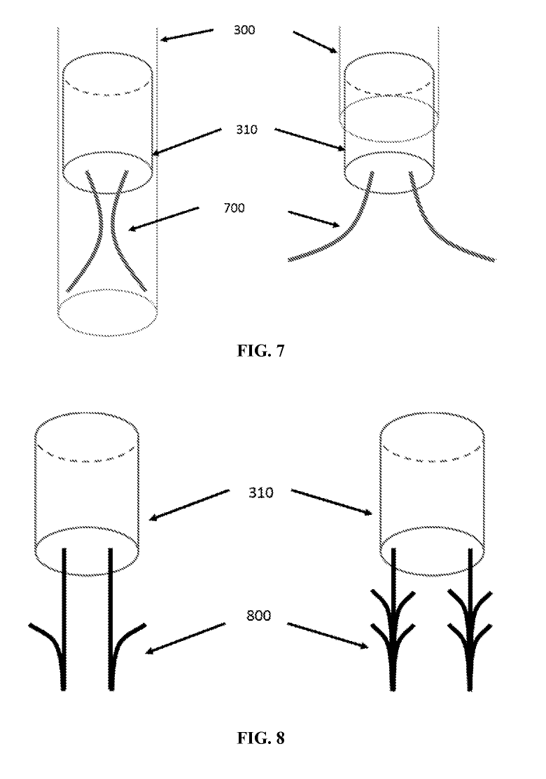

The implant main body 310, together with any of a variety of antennas (a few examples are 520, 620, 640, or 660), may be positioned close to the surface of the tissue by first securing the electrodes at the desired location and then ejecting the device from the needle while the needle is being pulled back. By having electrodes attached to the main body 310 using flexible wires or leads 700, the main body 310 can stay in the needle or injection device 300 while the wires or leads 700 are unraveling as the needle is being pulled back. This is illustrated in FIG. 7. Then at the desired position, the remaining part of the implantable device can be ejected from the needle.

The antenna can be unfolded or unrolled using inflation (e.g. via a fluid such as a gas, a liquid and/or a gel) and/or by incorporating spring-like structures into the antenna material, such that it naturally expands into the usable shape after it is ejected from the needle. In case of inflatable antenna, the needle and the injection mechanism can have a small inflation tube 530 that is connected to the flexible antenna substrate. Once the implant with the folded antenna is ejected from the needle, the inflation mechanism engages, thus inflating the flexible substrate antenna and thereby unfolding it into a usable shape. Once unfolded, the excess inflation gas or liquid (such as saline) can be optionally evacuated, and the inflation tube can be severed from the antenna.

Electrodes 320 can be connected to the main body 310 as simple extensions and can further be made expandable after ejection from the needle. This can benefit the electrode configuration and placement, which can be adapted based on the needs of the application. The electrodes can be made from semi-rigid material and be fabricated such that they expand like a spring, tweezer-like element, or other expandable structure 700. Because of their flexibility, when placed inside the needle, they would naturally be compressed and therefore easily delivered with a desired separation. This process results in a larger separation than needle otherwise allows as shown in FIG. 7.

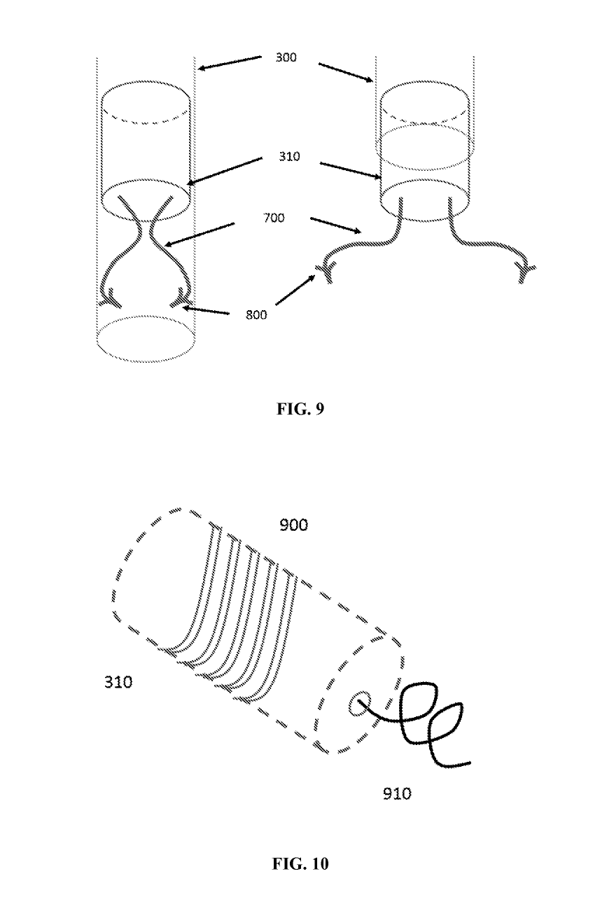

It may be beneficial to fix the device in position to prevent it from moving around and potentially for better electrode/tissue contact for some implantable devices. In this case, electrodes can include an anchor element 800, and can include a harpoon-shaped element 800; an arrow-shaped element 800; and/or threads, as shown in FIG. 8 and FIG. 9. The barbed electrodes 800 puncture and attach to tissues and fix the device in place. These configurations would penetrate the desired tissue or organ and prevent it from being detached from it. The barbed electrodes 800 can also be used at the tip of the flexible electrodes 700 that expand upon delivery for better or more precise placement of the interface. This also allows for a simpler implantation procedure without any stitching or otherwise more complex attachment procedure.

Another possible attachment that allows for easy implantation is by incorporating a screw-like electrode on the device as shown in FIG. 10. The screw electrode 910 screws into tissue to fix it in place. The screwing motion can be accomplished with threading 900 on the implant body 310 that has a matching thread on the delivery needle 300. As the implant body 310 is pushed out of the needle, it will rotate due to the threading. The device can then screw into tissue for a better electrode-tissue interface and a secure attachment. Implant body 310 can contain a counter-electrode of opposite polarity to the active electrode 910 when electrode is being used to modulate tissue. Alternatively, the self-attaching structures (800 and 910) can simply be used to fixate the implant 310 to the target tissue.

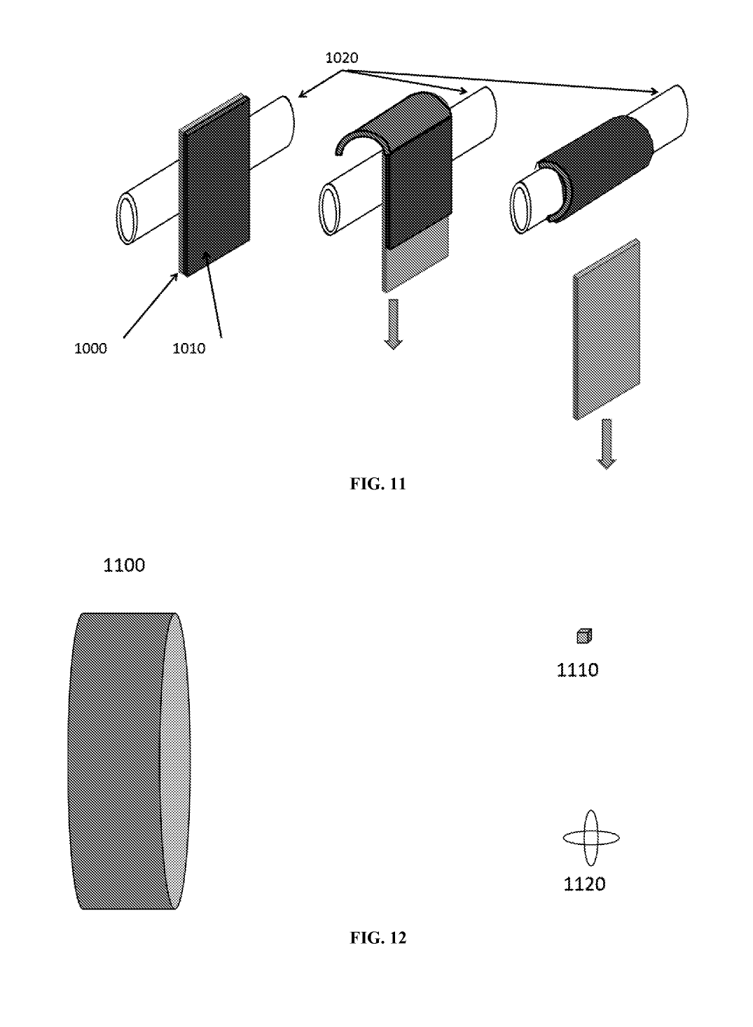

In some other procedures or applications it may be necessary to fix the implantable device around an organ or tissue, such as a vein or an artery. This can be accomplished by making the entire implantable device or some part of it cuff around that organ as shown in FIG. 11. This figure shows a rigid surface 1000 and a bendable or pre-formed substrate 1010. The mechanism for wrapping this system around the target site 1020 is shown. This can be accomplished in several different ways, such as two sliding beams, with one beam (rigid beam 1000) staying in the delivery system 300 and the other (compliant beam 1010) bending around the site 1020 that needs to be treated or diagnosed. As the comformable beam 1010 is ejected from the introducer 300, the rigid beam 1000 separates from the comformable beam 1010 and the comformable beam 1010 curves, naturally wrapping around target organ 1020. Another way is to compress a curved substrate 1010 into the needle that would serve as the rigid surface 1000, which would remain straight until ejected, and then would curve into a tight “ring” around the vein or artery as shown in FIG. 11. This method of action would be substantially similar to a slap (snap) bracelet (“Slap Wrap”). This method would contain a bi-stable spring which can be straightened out for delivery. The straightened bracelet cuff could then wrap around the target tissue 1020, forming a tight ring. This is caused by having the bi-stable spring transition into the second stable state (curved state), securing the implant 110 at the target site 320.

In one embodiment, the injection apparatus can consist of the main body 310 and a needle or introducer 300. The needle can be made rigid, flexible, or a combination to accommodate a particular implantation procedure. If it may be necessary to navigate around certain areas or organs, the bending or flexible needle can navigate around them and thus avoid certain locations within the body of tissue. This may also be beneficial if the implantable device needs to be positioned at a particular angle that is preferential over a straight line.

In one embodiment, an implantation procedure may involve: 1) injection of the needle or introducer 300 and its navigation to the implantation site; 2) initial ejection of the device 110 from the needle 300 until the electrodes are in contact with the stimulation site; 3 a) if the device is fully contained without any extensions, then complete the delivery process of the device; 3b) if the device has one or more extensions, then proceed with retraction of the needle where the extension or the leads are unwinding at the same rate while the needle is being extracted (pulled back) from the implantation site; 4) if unfolding or unrolling of the extensions is necessary, proceed with the unfolding procedure once the unfolding extension (510, 610, 630, 650) is ejected from the needle; 5) once the device and all of the optional extensions are fully ejected, remove the needle or introducer 300.

The injection device can further be used for extraction of the implant, if there may be a need. The needle or introducer 300 can be inserted and positioned to come in contact with the device and attach to it with suction forces. The extraction device can be used to extract the entire implant or a part of it, such as a particular extension. This can be done to replace or remove certain parts that can degrade or become unnecessary over time. In that case, the extensions can be made detachable if need be. Alternatively, there may be a temporary (dissolvable) or permanent thread attached to the device, which can extend to the outside the body or be left close to the skin surface in the subcutaneous surface. This way, the device can be removed easily by pulling on the thread.

Both injection and extraction of the implantable device can be guided using some sort of existing imaging modality from outside of the body of tissue, such as ultrasound, X-Ray, MRI, etc. Alternatively, it can be guided by optical fiber or a camera that can be placed at the tip of the injection needle. Furthermore, a technique similar to laparoscopy can be used for the implantation of the device. Conventional guidance methods such as catheters or non-invasive procedures may also be used.

The implant can also be covered with biocompatible, bio-dissolvable, biodegradable materials. In some embodiments, the electrodes may be encapsulated with a bio-dissolvable compound, which is present for ease of implantation, but is dissolved over a period of time post injection. Additionally or alternatively, the electrodes may be coated with drug eluting materials for proper adaptation and to minimize infection, inflammation, and rejection risks.

The needle injectable implant can be packaged in a sterile needle and packaged together with a disposable injection device. The needle and the injection device may be similar to currently available needles and syringes and can be customized for a particular therapy application.

Neuromodulation devices can require significant power to provide therapy, because of the relatively high voltage and current requirements needed to drive stimulation. Essentially, mechanical neuromodulation provides a more efficient conversion from electrical energy to therapeutic stimulation, and offers additional advantages in terms of safety and long-term use. Mechanically activating nerves can be accomplished with very small devices and with power-efficient mechanisms for generating force. Using electromagnetic forces allows for controllable stimulation with a fraction of the voltage and current requirements of electrical stimulation, and requires no direct electrical connection with tissue. This allows the device to be entirely encapsulated in bio-compatible materials if needed, and can therefore operate safely for extended periods of time. Depending on the needs of the application, this actuation can be accomplished by generating a magnetic field to control a magnetic material, by controlling currents in a preexisting magnetic field, or with some combination of the two. The force from these mechanical actuators can be applied directly to nerves or other tissues to activate them with similar characteristics as electrical stimulation, which results in similar therapeutic outcomes. This mechanical modulation system can be incorporated in the implant body 310 or it can be a separate extension as described. It can also be used in combination with sensors or other extensions, and it may be advantageous to have mechanical modulation operating in conjunction with electrical modulation.

As described, electromagnetic forces can be used in two different ways. The first method manipulates a magnet or a magnetic material by adjusting the magnetic field. This field can be adjusted with permanent magnets or electromagnets. The force on the material is given by

Fi=Vₘ(MV)B

where Fi is the force, Vₘ is the volume of the material, M is the magnetization of the material, and B is the strength of the magnetic field. These forces are proportional the gradient of the field and the volume of the structure, though can be quite large even at small sizes. The second force is exerted on currents flowing in the presence of a magnetic field, and is described by the Lorentz force which is

F₂=I(L×B)

where F₂ is the force, I is the current flowing through the wire, L is the length of the wire, and B is the magnetic field strength. With strong magnetic fields, large forces can be generated with relatively low currents, making this force very power efficient. This force can be amplified with additional loops of wire. The magnetic field can be generated with permanent magnets or electromagnets, and be either external or internal to the body.

To control the motion of a magnetic material, an electromagnet can be used to adjust the strength of the magnetic field. An effective way of accomplishing this is with several loops of wire around either a magnet or a magnetic material, similar to a solenoid. Current flows through the coil, inducing a magnetic field which applies a force to the magnet or magnetic material inside. This current can be oscillated to produce oscillating forces. The magnetic field generated in the center of the coil by this device is

B=μₒ(N/L)I

where B is the magnetic field, μo is the magnetic permeability, N is the number of loops in the coil, L is the length of the coil, and I is the current flowing through the coil. The force on the magnetic material is given by Fi and depends on the size of the material and its magnetization. Ferrite can have relative permeability −1000, and mu-metals can have relative permeability of −50,000 or more. Using a material with a high relative permeability allows for mm-sized and sub-mm devices to exert large enough forces to activate nerves. These devices can be electrically powered from an implantable device using 10-1OO× less power than direct electrical stimulation. The magnetic material in this method does not need to be located in the center of the coil, it only needs to be in range of the generated magnetic field to experience a force.

Additionally, the restoring force can be supplied mechanically such as with a spring so that the electromechanical force is only exerted in one direction. This is especially useful if a magnetic material is used because the force is insensitive to the direction of the field, it is only sensitive to the gradient.

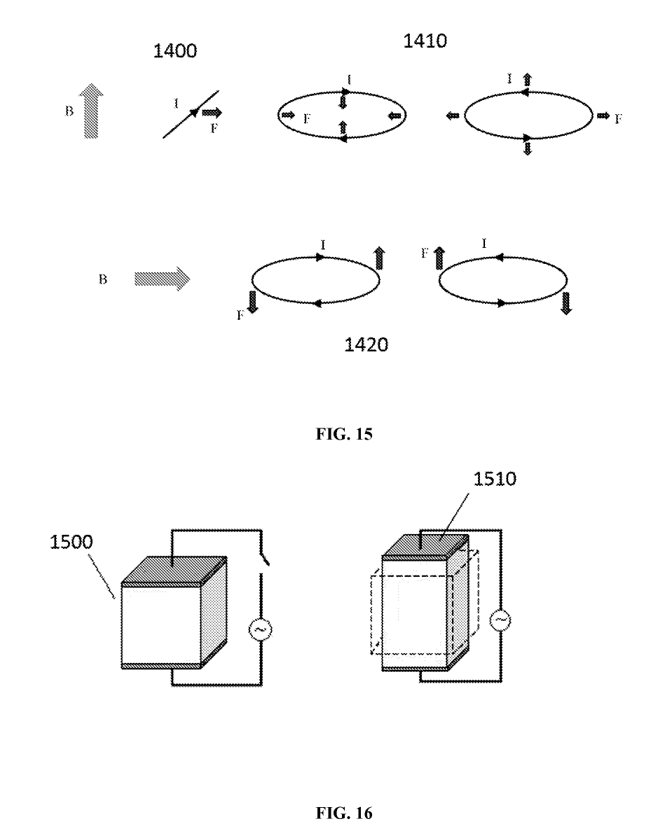

An alternative method employs the forces exerted on current-carrying wires to actuate nerves. This method requires a magnetic field to be present, which can be provided from either a permanent magnet or an electromagnet, which can be either external or internal to the body. The force experienced by the wires is given by F₂ and is proportional to the magnetic field and the current. Additional wires can also be used to magnify the force. The current flowing in the wires can be controlled to precisely control the force on them, and several arrangements of wires can surround the nerves to apply a variety of forces to best activate the target nerve. These forces can squeeze, expand, push, or pull the nerve as needed. Several possible arrangements are shown in FIG. 15. This figure shows wires that generate linear forces 1400, loops that experience compression or expansion forces 1410, and loops that experience torques 1420. These arrangements show how several types of forces and torques can be generated. A multitude of wire arrangements are possible, and the actuator can be designed for the targeted nerve with one or more of them. The current in the wires can oscillate with controlled parameters, such as frequency, amplitude, and duty cycle, to induce the desired mechanical forces.

An example of an implanted material experiencing forces due to Fi and F₂ is shown in FIG. 12. The magnetic field source/generator 1100 is outside of the body and induces forces on the implanted magnetic material 1110 or on current carrying wire arrangements 1120 through the use of magnetic fields. These forces can cause vibrations, rotations, or linear motion of the implanted material. If the material is attached or in the vicinity of targeted tissues, it can mechanically modulate them.

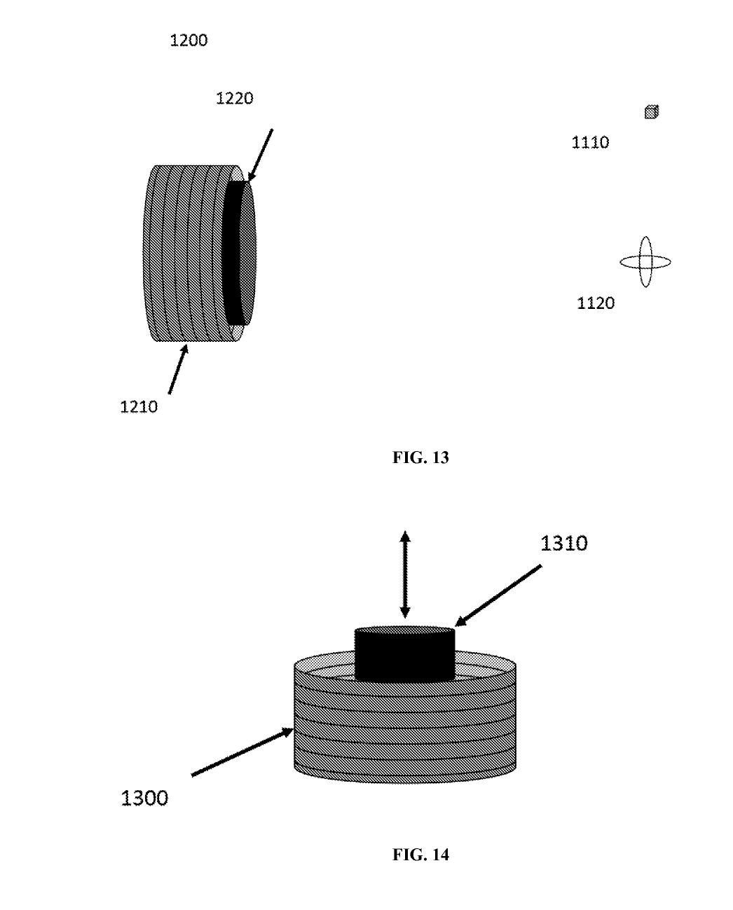

An example of an implanted material experiencing forces due to Fi and F₂ using magnetic fields from an electromagnet is shown in FIG. 13. The electromagnet 1200 is outside of the body and induces forces on the implanted magnetic material 1110 or on current carrying wire arrangements 1120 through the use of magnetic fields. The electromagnet 1200 includes coils of wire 1210 and an optional magnetic structure 1220 to enhance the generated field. These forces can cause vibrations, rotations, or linear motion of the implanted material 1110. If the material is attached or in the vicinity of targeted tissues, it can mechanically modulate them.

Alternatively, the implant main body 310 or an extension may include a miniaturized electromagnet, as depicted in FIG. 14. This electromagnet again consists of a smaller arrangement of coils of wire 1300 and a movable magnetic structure 1310. The field caused by currents flowing in the coils of wire 1300 induces forces on the magnetic structure 1310, causing it to move. The currents can be controlled to create controllable motion. These forces can cause vibrations that modulate surrounding tissues mechanically.

For some applications, it may be desirable to generate magnetic fields externally to reduce the complexity of the actuators, to allow for MRI-compatibility if permanent magnets are required, or for miniaturization. This can be accomplished with either an electromagnet or a permanent magnet. This field generation can be co-designed with a power and data transmitter if necessary or can be a separate component of the system. These magnetic fields are generated in the proper direction for the desired force, and higher field strengths result in improved power efficiency, as is evident from the force equations. Electromagnets can be enhanced with high permeability materials, which magnify the field strength.

For either mechanical actuation method, the forces must be designed to stimulate the nerves in a therapeutic way. A key advantage of electromagnetic methods is the ability to operate at a variety of frequencies and to have a highly adjustable force that operates effectively as the device is miniaturized. The stimulation can be optimized in terms of frequency, force, and duty cycle, simply by adjusting the flowing currents. For instance, some nerves are best stimulated with short bursts of high-frequency vibrations, while other nerves are best stimulated with one strong, high force pulse. In the case of fully wireless implants including battery-free, remotely powered devices, this means that the stimulation characteristics can be completely controlled externally with commands from the transmitter and have significant flexibility in operation. Additionally, the scalability of the forces allows for mm and sub-mm operation, allowing for use with devices that can be injected with a needle, placed with an endoscope, or placed with a catheter.

The actual size of the actuators is determined by the needs of the application, as larger nerves may require more force for controlled stimulation. Some nerves, such as baroreceptors, are sensitive to the stretching of blood vessels, and so would require a mechanical structure to apply this type of force. Other nerves can be excited by localized tapping with certain parameters, and would require a different mechanical structure. The arrangements shown in FIG. 15 depict how these types of forces can be accomplished with the described actuators. From the force equations, it is clear that the described force actuation mechanisms have the power efficiency and scalability to meet the needs of these different applications, and the versatility to apply a variety of different forces at different sizes.

Because the forces are easily controlled by manipulating flowing currents, these mechanical methods are easily integrated into existing devices, including fully wireless devices. They can also be implemented on a passive device, where the actuation is due to the manipulation of a magnetic structure with externally applied magnetic fields. For powered devices, the interface circuitry can be designed so that the stimulator controller is either analog or digital. Digital control offers simplicity and more universal operation, though analog control could be better optimized for increased power efficiency. The mechanical stimulators can operate with similar waveforms to that of electrical stimulators, and can therefore serve as a replacement with minimal alteration to existing devices. Additionally, these mechanical techniques can have significant advantages in terms of power efficiency and long-term safety because the tissue impedance does not impact performance and no electrical connection is required. Their high power-efficiency allows for use with remotely powered devices, which have a very limited power budget. This power efficiency would also prolong battery life for conventional devices. The level of miniaturization and efficiency also allows for entirely new devices which may be small enough for non-invasive implantation as previously discussed.

When implanted in the body, these mechanisms can be packaged with epoxies or plastics that encapsulate it for bio-compatibility. These epoxies are ready available, and similar materials can be used to that of existing medical devices. Because no electrical connection is required, the device can be fully encapsulated and protected from the body without hindering performance. The forces will be experienced by the actuators regardless of the surrounding material, as common encapsulating materials are effectively transparent to electric and magnetic fields.

For some therapies, other types of mechanical actuators may be better suited for activating nerves. Piezoelectric materials, such as those shown in FIG. 16, can apply large forces if they are needed, and can constructed in the form of vibrating beams, motors, linear actuators, or other vibrating structures designed with specific resonances. They operate when the piezoelectric 1500 has a voltage applied through an electrical contact 1510, thus inducing mechanical deformation. These devices can be efficient when operating at resonance, and can apply large forces though the forces get fairly weak as they are scaled down to mm-scales.

Additionally, thermoelectric actuators could be useful for small devices and would not require the presence of a magnetic field.

Although the present invention has been particularly described with reference to embodiments thereof, various changes, modification and substitutes are intended within the form and details thereof, without departing from the spirit and scope of the invention. Accordingly, in numerous instances, some features of the invention will be employed without a corresponding use of other features. Further, variations can be made in the number and arrangement of components illustrated in the above figures.

1. A medical apparatus for interfacing with tissue of a patient comprising:
an implantable system comprising:
a body portion for implanting within a patient; and
at least one extension attached to the body portion;
an external system that transcutaneously transmits at least one of power or data to the implantable system during operation; and
an introducer for delivering the body portion and the at least one extension into the patient.

2. The medical apparatus according to claim 1, wherein the medical apparatus is configured to induce a physiologic response in tissue.

3. The medical apparatus according to claim 1, wherein the medical apparatus is configured to monitor tissue activity.

4. The medical apparatus according to claim 1, wherein the external system is configured to transcutaneously transmit both power and data to the implantable system during operation.

5. The medical apparatus according to claim 1, wherein the implantable system performs a function selected from the group consisting of: delivering energy to tissue; delivering therapy to tissue; delivering pharmaceutical agent to tissue; modulating tissue;
sensing physiological activity; sensing neural activity; sensing environmental conditions; sensing therapeutic outcomes; sensing delivered therapy parameters; and
combinations thereof.

6. The medical apparatus according to claim 1, wherein the at least one extension comprises an element selected from the group consisting of: a lead; an electrode; a conformable electrode; two sliding beams; a cuff electrode; an anchor element; an antenna; an energy storage element; an energy harvesting element; and combinations thereof.

7. The medical apparatus according to claim 1, wherein the implantable system further comprises an element selected from the group consisting of: a tissue modulator; an actuator; an electromagnetic actuator; a sensor; and combinations thereof.

8. The medical apparatus according to claim 1, wherein the implantable system further comprises a harvesting element constructed and arranged to harvest energy from the patient, wherein the harvested energy comprises energy selected from the group consisting of: heat energy; motion energy; chemical energy; and combinations thereof.

9. The medical apparatus according to claim 1, wherein the implantable system comprises a power receiver configured to receive energy selected from the group consisting of: radiofrequency energy; ultrasound energy; and combinations thereof.

10. The medical apparatus according to claim 1, wherein the implantable system further comprises an energy storage element selected from the group consisting of: a battery; a capacitor; and combinations thereof.

11. The medical apparatus according to claim 1, wherein the at least one extension is constructed and arranged to transition from a compacted state to an expanded state, and wherein the transition comprises a physical change selected from the group consisting of: unfolding; unrolling; unfurling; and combinations thereof.

12. The medical apparatus according to claim 1, wherein the at least one extension comprises an inflatable portion constructed and arranged to expand when filled with fluid selected from the group of: a gas; a liquid; a gel; and combinations thereof.

13. The medical apparatus according to claim 1, wherein the at least one extension comprises an anchor element selected from the group consisting of: threads;
arrow-shaped element; harpoon-shaped element; and combinations thereof.

14. The medical apparatus according to claim 1, wherein the at least one extension is constructed and arranged to detach from the body portion.

15. The medical apparatus according to claim 1, wherein the introducer comprises a flexible cannula and a rigid cannula, and wherein the flexible cannula is constructed and arranged to slidingly receive the rigid cannula.

16. The medical apparatus according to claim 1, wherein the body portion and at least one extension are constructed and arranged to exit the introducer while the introducer is being removed from the body of the patient.

17. The medical apparatus according to claim 1, further comprising a sealing housing, wherein the body portion is contained in the housing.
